(19) United States
(12) Patent Application Publication
(10) Pub. No.: US 2016/0369123 A1
(43) Pub. Date: Dec. 22, 2016

(54) COATING AGENT COMPOSITION AND UTILIZATION OF SAME

(71) Applicant: SUMITOMO BAKELITE CO., LTD., Shinagawa-ku, Tokyo (JP)

(72) Inventors: Wataru TAKADA, Tokyo (JP); Takayuki MATSUMOTO, Tokyo (JP); Sohei FUNACKA, Tokyo (JP)

(73) Assignee: SUMITOMO BAKELITE CO., LTD., Shinagawa-ku, Tokyo (JP)

(21) Appl. No.: 15/117,794

(22) PCT Filed: Mar. 20, 2015
(86) PCT No.: PCT/JP2015/058557

§ 371 Date: Aug. 10, 2016

(30) Foreign Application Priority Data
Mar. 31, 2014 (JP) 2014-071194

(51) Int. Cl. C09D 133/14; G01N 33/543; C08F 220/28; C08J 7/04; C08J 7/12

(52) U.S. Cl. CPC C09D 133/14; C08J 7/047; C08J 7/12; C08F 220/28; G01N 33/54353; C08J 2365/00; C08J 2433/14; C08F 2220/286

(57) ABSTRACT

This coating agent composition is characterized by including: a copolymer (A) having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking, and a copolymer (B) having a repeating unit derived from the monomer (a); and a repeating unit derived from the monomer (b), and having a reactive functional group at the terminal of at least one side of the copolymer, and is used for coating the surface of a solid-phase substrate.

### TECHNICAL FIELD

The present invention relates to a polymer compound for a medical material having a function of immobilizing a biologically active substance. Furthermore, the present invention relates to a surface coating agent including the polymer material, and a biodevice making use of the polymer compound.

### BACKGROUND ART

Attempts to evaluate gene activity or to decode the physiological processes of drug effects at a molecular level have been traditionally focused on genomics. However, proteomics provides more detailed information on the biological functions of cells. Proteomics includes qualitative and quantitative analysis of gene activity based on detection and quantification of the expression at the protein level, rather than expression at the gene level. Proteomics also includes a study of phenomena that are not encoded in genes, such as modification after translation of a protein, and interaction between proteins.

Furthermore, in recent years, attention has been paid to sugar chain molecules as a third class of chain subsequent to nucleic acids and proteins, and study termed glycomics is ongoing in accordance with genomics and proteomics. Particularly, research is being conducted in relation to cell differentiation or canceration, immune reactions, insemination, and the like, and attempts to create new medicines or medical materials are being made continuously.

Furthermore, sugar chains are receptors for many toxins, viruses, bacteria, and the like, and attention has also been paid to sugar chains as cancer markers. Thus, also in these fields, attempts to create new medicines or medical materials are similarly continuing.

Today, since a large amount of genome information is available, there is a further demand for increased rapidity and increased efficiency (high throughput) in connection with the detection of physiologically active substances. As a molecular array intended for this purpose, DNA chips have been put to practical use. On the other hand, in regard to the detection of proteins or sugar chains that are complicated and highly diverse in biological functions, protein chips and sugar chain chips have been proposed, and research has been in progress in recent years.

Since current protein chips are generally regarded as an extension of DNA chips, and development thereof has been achieved from this point of view, investigations have been conducted on the issue of immobilizing proteins, or molecules that trap the proteins, on a solid-state surface of a glass substrate plate or the like (for example, Patent Document 1).

Meanwhile, it has been recognized in many cases that sugar chains function as ligands for cell receptors, rather than exhibiting their functions by themselves. Therefore, in order to supply sugar chains for analyses of receptors in connection with the sugar chains, development of base materials intended for immobilization of various sugar chains has also been carried out (for example, Patent Document 2).

In regard to the signal detection of a protein chip or a sugar chain chip, one factor that decreases the signal-to-noise ratio may be the non-specific adsorption of a substance to be detected, to a substrate plate (see, for example, Non-Patent Document 1).

In a case in which such a biochip is used, there is a problem that during a washing process after capturing a protein or a sugar chain, the protein or sugar chain that has been immobilized on the substrate plate, and the molecule that captures the protein or sugar chain may flow out, and the signal may decrease. As an approach to address this problem, a method of forming a functional surface that is strongly bonded onto a support by applying an active component containing a functional group, a spacer group and a binding group, a crosslinking component, and a matrix-forming component on the support, and curing the components, has been disclosed (for example, Patent Document 3).

### CITATION LIST

### Patent Literature

[Patent Document 1] Japanese Unexamined Patent Application, First Publication No. 2001-116750
[Patent Document 2] Japanese Unexamined Patent Application, First Publication No. 2004-115616
[Patent Document 3] Published Japanese Translation No. 2004-531390 of the PCT International Publication

### Non-Patent Literature

[Non-Patent Document 1] “Practical DNA Microarray Manual”, edited by HAYASHIZAKI, Yoshihide and OKAZAKI, Yasushi, Yodosha Co., Ltd., 2000, p. 57

### SUMMARY OF INVENTION

### Technical Problem

The present disclosure provides, in one or plural embodiments, a coating agent that can form a biochip having an increased S/N ratio.

Two kinds of method have been carried out as a method for immobilizing a physiologically active substance. One of them is a method of achieving immobilization based on physical adsorption of a protein. In this method, a coating with an adsorption inhibitor is performed in order to prevent non-specific adsorption of secondary antibodies after a protein is immobilized; however, the non-specific adsorption prevention ability of these adsorption inhibitors is not sufficient. Furthermore, since a coating with the adsorption inhibitor is performed after primary antibodies are immobilized, the adsorption inhibitor is applied on immobilized proteins, and there is a problem that the biochip and the secondary antibodies cannot react. Therefore, there is a demand for a biochip which does not need coating with an adsorption inhibitor after immobilization of primary antibodies and has a small amount of non-specific adsorption of a physiologically active substance.

In the method disclosed in Patent Document 3 described above, since curing of low molecular weight components proceeds on the support, in a case in which the support is a plastic substrate plate, there is a risk that warpage or deformation may occur. Furthermore, since a matrix that is intertwined into a network form is formed, there is a problem that the reaction of functional groups for immobilizing a physiologically active substance may be suppressed, or the reproducibility of the expression of functions of the immobilized physiologically active substance may be poor. Furthermore, since it is difficult to wash the proteins that have penetrated into the matrix, non-specific adsorption may not be sufficiently suppressed.

### Solution to Problem

That is, the present invention is as follows.

(1) A coating agent composition used to coat the surface of a solid-phase substrate comprising: (A) a copolymer having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking, and a copolymer (13) having a repeating unit derived from the monomer (a); and a repeating unit derived from the monomer (b), and having a reactive functional group at the terminal of at least one side of the copolymer.

(2) A coating agent kit used to coat the surface of a solid-phase substrate comprising: a copolymer (A) having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking, and a copolymer (B) having a repeating unit derived from the monomer (a); and a repeating unit derived from the monomer (b), and having a reactive functional group at the terminal of at least one side of the copolymer, wherein the copolymer (A) and the copolymer (B) are respectively accommodated in different containers.

(3) A method for producing a solid-phase substrate having a coated surface comprising: a step of applying a coating agent composition on the surface of a solid-phase substrate, the coating agent composition including: a copolymer (A) having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking, a copolymer (B) having a repeating unit derived from the monomer (a); and a repeating unit derived from the monomer (b), and having a reactive functional group at the terminal of at least one side of the copolymer, and a solvent; and a step of removing the solvent from the coating agent composition applied on the solid-phase substrate.

(4) A method for producing a solid-phase substrate having a coated surface comprising: a step of applying a first coating agent composition on the surface of a solid-phase substrate, the first coating agent composition including: a copolymer (A) having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking, and a solvent; a step of removing the solvent from the first coating agent composition applied on the solid-phase substrate, and obtaining a solid-phase substrate having the surface coated with the copolymer (A); a step of applying a second coating agent composition on the surface of the solid-phase substrate having the surface coated with the copolymer (A), the second coating agent composition including: a copolymer (B) which has a repeating unit derived from the monomer (a); and a repeating unit derived from the monomer (b), and has a reactive functional group at the terminal of at least one side of the copolymer, and a solvent; and a step of removing the solvent from the second coating agent composition applied on the solid-phase substrate.

(5) A solid-phase substrate having a surface coated with: a copolymer (A) having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking; and a copolymer (B) having a repeating unit derived from the monomer (a); and a repeating unit derived from the monomer (b), and having a reactive functional group at the terminal of at least one side of the copolymer.

(6) A biosensor comprising a physiologically active substance immobilized on the solid-phase substrate according to (5).

(7) A method for producing the biosensor according to (6) comprising a step of immobilizing a physiologically active substance on the solid-phase substrate according to (5).

<1> A coating agent used to coat the surface of a solid-phase substrate,

in which the coating agent includes a copolymer (A) and a copolymer (B),

the copolymer (A) and the copolymer (B) both are copolymers including a constituent unit derived from (a) an ethylenically unsaturated polymerizable monomer having one ethylenic double bond and an alkylene glycol residue; and a constituent unit derived from (b) an ethylenically unsaturated polymerizable monomer having one ethylenic double bond and a functional group for immobilizing a physiologically active substance,

the copolymer (A) further includes a constituent unit derived from (c) an ethylenically unsaturated polymerizable monomer having one ethylenic double bond and a functional group capable of crosslinking, and

the copolymer (B) has a reactive functional group at the terminal of at least one side of the copolymer.

<2> The coating agent according to <1>, in which the functional group for immobilizing physiological activity is an active ester group.

<3> The coating agent according to <1> or <2>, in which the functional group capable of crosslinking is an alkoxysilyl group.

<4> The coating agent according to any one of <1> to <3>, in which the reactive functional group is an alkoxysilyl group.

<5> The coating agent according to any one of <1> to <4>, in which the copolymer A and the copolymer B exist in the form of a mixture.

<6> The coating agent according to any one of <1> to <5>, in which the copolymer A and the copolymer B are respectively accommodated in different containers.

<7> The coating agent according to any one of <1> to <6>, in which the (a) ethylenically unsaturated polymerizable monomer having one ethylenic double bond and an alkylene glycol residue is a monomer represented by the following General Formula [1].

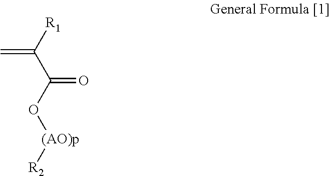

(In the formula, R₁ represents a hydrogen atom or a methyl group; R₂ represents a hydrogen atom, a methyl group, or an ethyl group; AO represents an alkylene oxide group having 2 to 10 carbon atoms; and p represents the average number of added moles of AO and is a number of 1 to 100.)

<8> The coating agent according to any one of <1> to <7>, in which the (b) ethylenically unsaturated polymerizable monomer having one ethylenic double bond and a functional group for immobilizing a physiologically active substance is a monomer represented by the following General Formula [2] and having an active ester.

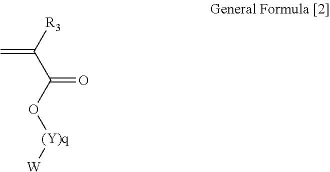

(In the formula, R₃ represents a hydrogen atom or a methyl group; Y represents AO or an alkyl group having 1 to 10 carbon atoms (q=1); AO represents an alkylene oxide group having 2 to 10 carbon atoms; q represents the average number of added moles and is a number of 1 to 100; and W represents an active ester group.)

<9> The coating agent according to any one of <1> to <8>, in which the constituent unit derived from (c) an ethylenically unsaturated polymerizable monomer having one ethylenic double bond and a functional group capable of crosslinking is a monomer represented by the following General Formula [3]:

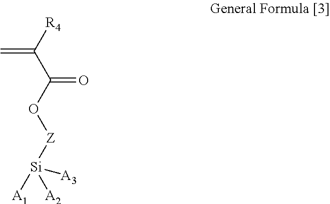

(In the formula, R₄ represents a hydrogen atom or a methyl group; Z represents an alkyl group having 1 to 20 carbon atoms; and at least one of A₁, A₂ and A₃ represents a group capable of hydrolysis, while the others represent inactive groups that are not hydrolyzable.)

<10> The coating agent according to any one of <1> to <9>, in which the copolymer A is represented by the following General Formula [4].

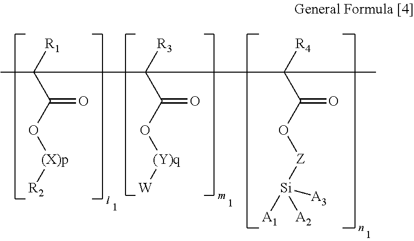

(In the formula, R₁ represents a hydrogen atom or a methyl group; R₂ represents a hydrogen atom, a methyl group, or an ethyl group; AO represents an alkylene oxide group having 2 to 10 carbon atoms; p represents the average number of added moles of AO and is a number of 1 to 100;

R₃ represents a hydrogen atom or a methyl group; Y represents AO or an alkyl group having 1 to 10 carbon atoms (q=1); AO represents an alkylene oxide group having 2 to 10 carbon atoms; q represents the average number of added moles of AO and is a number of 1 to 100; W represents an active ester group;

R₄ represents a hydrogen atom or a methyl group; Z represents an alkyl group having 1 to 20 carbon atoms; at least one of A₁, A₂ and A₃ represents a group capable of hydrolysis, while the others represent inactive groups that are not hydrolyzable;

the proportion of l₁ with respect to the sum of l₁, m₁ and n₁ is 5 to 98 mol %, the proportion of m₁ with respect to the sum of l₁, m₁ and n₁ is 1 to 94 mol %; and the proportion of n₁ with respect to the sum of l₁, m₁, and n₁ is 0.01 to 30 mol %.)

<11> The coating agent according to any one of <1> to <10>, in which the copolymer B is represented by the following General Formula [5].

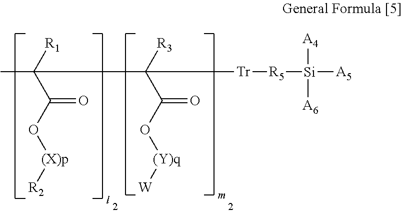

(In the formula, R₁ represents a hydrogen atom or a methyl group; R₂ represents a hydrogen atom, a methyl group, or an ethyl group; AO represents an alkylene oxide group having 2 to 10 carbon atoms; p represents the average number of added moles of AO and is a number of 1 to 100;

R₃ represents a hydrogen atom or a methyl group; Y represents AO or an alkyl group having 1 to 10 carbon atoms (q=1); AO represents an alkylene oxide group having 2 to 10 carbon atoms; q represents the average number of added moles of AO and is a number of 1 to 100; W represents an active ester group;

R₅ represents a hydrocarbon chain having 1 to 20 carbon atoms, which may be interrupted by —O—, —S—, —NH—, —CO—, or —CONH—; at least one of A₄, A₅ and A₆ represents a group capable of hydrolysis, while the others represent inactive groups that are not hydrolyzable;

the proportion of l₂ with respect to the sum of l₂ and m₂ is 5 to 98 mol %; the proportion of m₂ with respect to the sum of l₂ and m₂ is 1 to 94 mol %; and

Tr represents a group derived from a chain transfer agent.)

<12> A solid-phase substrate having a surface coated with the coating agent according to any one of <1> to <11>.

<13> A biosensor having a physiologically active substance immobilized on the solid-phase substrate according to <12>.

<14> A method for producing a solid-phase substrate, the method including a step of bringing the surface of a solid-phase substrate into contact with the coating agent according to any one of <1> to <11>, and treating the surface of the solid-phase substrate.

<15> A method for producing the biosensor according to <14>, the method further including a step of immobilizing a physiologically active substance on the solid-phase substrate.

### Advantageous Effects of Invention

According to the invention, a biodevice having a high S/N ratio can be provided in one or a plurality of embodiments.

### DESCRIPTION OF EMBODIMENTS

A protein chip is a generic name for chips (minute substrates) having a protein or a molecule that captures the protein, immobilized on the chip surface. A sugar chain chip is a generic name for chips having a sugar chain or a molecule that captures the sugar chain, immobilized on the chip surface.

In order to solve the problems described above, the inventors of the present invention have already invented two kinds of polymer compounds. Specifically, the invented polymer compounds are a copolymerized polymer compound of an ethylenically unsaturated polymerizable monomer having an alkylene glycol residue, an ethylenically unsaturated polymerizable monomer having a functional group for immobilizing a physiologically active substance, and an ethylenically unsaturated polymerizable monomer having a functional group capable of crosslinking, as described in Japanese Unexamined Patent Application, First Publication No. 2012-078365; and a copolymer derived from an ethylenically unsaturated polymerizable monomer having an alkylene glycol residue, and an ethylenically unsaturated polymerizable monomer having a functional group for immobilizing a physiologically active substance, as described in WO 2006/123737, the copolymer being a polymer compound having a reactive functional group at the terminal of at least one side of the polymer compound.

This time, the present inventors further conducted investigations, and finally developed a polymer compound which has superior immobilizing ability for a physiologically active substance than conventional compounds, and exhibits less non-specific adsorption to proteins and the like. The inventors found that these goals can be achieved by optimizing the compositions of two or more kinds of the polymer compounds described above, which have been previously invented by the inventors, and using the two kinds of polymer compounds as a mixture. Thus, the inventors completed the present invention.

In a case in which the two kinds of polymer compounds are respectively used alone, the reactive functional groups of the same polymer compound molecules undergo a (crosslinking) reaction; however, when the two polymer compounds are mixed, a (crosslinking) reaction also occurs between the different polymer compound molecules. It is speculated that thereby, a nature that is not obtainable solely by the way of physical entanglement of polymer chains (three-dimensional structure) is obtained, and this nature leads to the ease of contact between the functional groups that are responsible for immobilization of a physiologically active substance, and the physiologically active substance. Furthermore, in regard to the suppression of non-specific adsorption, it is also speculated that the property of suppressing non-specific adsorption that is exhibited by the respective polymer compounds can be retained.

[Coating Agent Composition]

According to an embodiment of the invention, there is provided a coating agent composition used to coat the surface of a solid-phase substrate comprising: a copolymer (A) having a repeating unit derived from (a) an ethylenically unsaturated polymerizable monomer having one ethylenic double bond and an alkylene glycol residue; a repeating unit derived from (b) an ethylenically unsaturated polymerizable monomer having one ethylenic double bond and a functional group for immobilizing a physiologically active substance; and a repeating unit derived from (c) an ethylenically unsaturated polymerizable monomer having one ethylenic double bond and a functional group capable of crosslinking, and a copolymer (B) having a repeating unit derived from the monomer (a); and a repeating unit derived from the monomer (b), and having a reactive functional group at the terminal of at least one side of the copolymer.

In other words, the copolymer (A) is a copolymer having a repeating unit represented by the following Formula (a), a repeating unit represented by the following Formula (b), and a repeating unit represented by the following Formula (c).

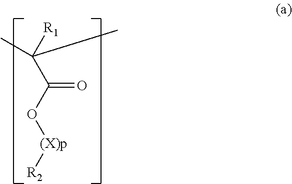

[In Formula (a), R₁ represents a hydrogen atom or a methyl group; R₂ represents a hydrogen atom, a methyl group, or an ethyl group; X represents an alkylene glycol residue having 2 to 10 carbon atoms; and p represents the number of repetitions of X and is a number of 1 to 100.]

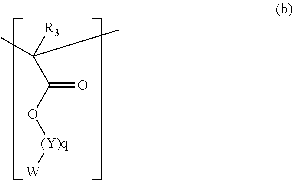

[In Formula (b), R₃ represents a hydrogen atom or a methyl group; Y represents AO or an alkylene group having 1 to 10 carbon atoms; AO represents an alkylene oxide group having 2 to 10 carbon atoms; q represents the average number of added moles of AO and is a number of 1 to 100; W represents an active ester group; and in a case in which Y represents an alkylene group, q=1.]

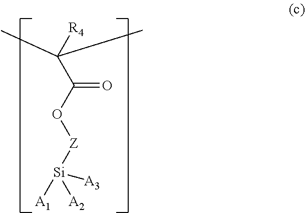

[In Formula (c), R₄ represents a hydrogen atom or a methyl group; Z represents an alkylene group having 1 to 20 carbon atoms; and at least one of A₁, A₂ and A₃ represents a group capable of hydrolysis, while the others represent inactive groups that are not hydrolyzable.]

Furthermore, the copolymer (B) is a copolymer having a repeating unit represented by Formula (a) described above, and a repeating unit represented by Formula (b) described above, and having a reactive functional group at the terminal of at least one side of the copolymer. Hereinafter, the copolymers (A) and (B) will be described in detail.

First of all, the two kinds of constituent units present in common in the copolymers (A) and (B) will be described in detail.

First, the constituent unit derived from (a) an ethylenically unsaturated polymerizable monomer having one ethylenic double bond and an alkylene glycol residue is not particularly limited in structure; however, it is preferable that the constituent unit is obtained by polymerizing a compound containing a chain of a compound represented by General Formula [1] having a (meth)acryl group and an alkylene glycol residue X having 1 to 10 carbon atoms.

According to the invention, the “alkylene glycol residue” means an “alkyleneoxy group” (—R—O—, where R represents an alkylene group) that remains after a condensation reaction between a hydroxyl group at the terminal of one side or the terminals of both sides of an alkylene glycol (HO—R—OH, where R represents an alkylene group), and another compound. For example, the “alkylene glycol residue” of methylene glycol (HO—CH₂—OH) is a methyleneoxy group (—CH₂—O—), and the “alkylene glycol residue” of ethylene glycol (HO—CH₂—CH₂—OH) is an ethyleneoxy group (—CH₂—CH₂—O—).

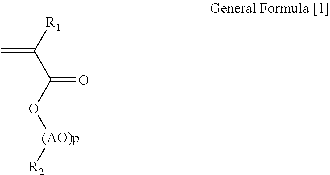

In General Formula [1], R₁ represents a hydrogen atom or a methyl group; R₂ represents a hydrogen atom, a methyl group, or an ethyl group; AO represents an alkylene oxide residue, which has 1 to 10 carbon atoms, preferably 1 to 6 carbon atoms, more preferably 2 to 4 carbon atoms, even more preferably 2 or 3 carbon atoms, and most preferably 2 carbon atoms. The number of repetitions p of the alkylene glycol residue X is not particularly limited to the average number of added moles of AO; however, the number of repetitions p is preferably an integer of 1 to 100, more preferably an integer of 2 to 100, even more preferably an integer of 2 to 95, and most preferably an integer of 3 to 90. In a case in which the number of repetitions p is from 2 to 100, the numbers of carbon atoms of the AO alkylene oxide residues that are repeated, may be identical or may be different.

Examples of the compound containing a chain of a (meth)acryl group and an alkylene glycol residue X having 1 to 10 carbon atoms include methoxy polyethylene glycol (meth)acrylate, ethoxy polyethylene glycol methacrylate; a (meth)acrylate of a monosubstituted ester of a hydroxyl group, such as 2-hydroxyethyl (meth)acrylate, 2-hydroxypropyl (meth)acrylate, or 2-hydroxybutyl (meth)acrylate; glycerol mono(meth)acrylate; a (meth)acrylate having polypropylene glycol as a side chain; 2-methoxyethyl (meth)acrylate; 2-ethoxyethyl (meth)acrylate; methoxy diethylene glycol (meth)acrylate; and ethoxy diethylene glycol (meth)acrylate. From the viewpoints of obtaining reduced non-specific adsorption of a physiologically active substance and availability methoxy polyethylene glycol methacrylate or ethoxy polyethylene glycol methacrylate is preferred.

Among them, methoxy polyethylene glycol (meth)acrylate or ethoxy polyethylene glycol (meth)acrylate, both having an average number of repetitions of ethylene glycol residues of 1 to 100, is preferably used from the viewpoint of having satisfactory operability (handleability) at the time of synthesis. Meanwhile, “(meth)acrylate” means methacrylate or acrylate.

Monomers (a) used for the copolymers (A) and (B) may be either monomers of the same kind, or monomers of different kinds.

Next, the constituent unit derived from (b) an ethylenically unsaturated polymerizable monomer having one ethylenic double bond and a functional group for immobilizing a physiologically active substance, is not particularly limited in structure; however, it is preferable that the constituent unit is obtained by polymerizing a compound represented by the following General Formula [2], the molecules of which are linked via a chain of a (meth)acryl group and an active ester group, the active ester group being an alkyl group or an alkylene glycol residue having 1 to 10 carbon atoms.

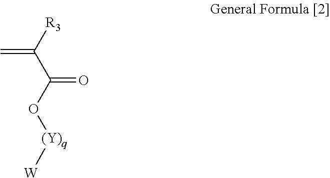

In General Formula [2], R₃ represents a hydrogen atom or a methyl group. Y represents (AO)q or an alkyl group having 1 to 10 carbon atoms, and AO represents an alkylene oxide group having 2 to 10 carbon atoms. It is preferable that Y is a chain of alkylene oxide groups each having 1 to 10 carbon atoms, or an alkyl group. In a case in which Y represents an alkylene oxide group, Y has 1 to 10 carbon atoms, preferably 1 to 6 carbon atoms, more preferably 2 to 4 carbon atoms, even more preferably 2 or 3 carbon atoms, and most preferably 2 carbon atoms. The number of repetitions q of the alkylene oxide group Y is an integer of 1 to 100, more preferably an integer of 2 to 90, and most preferably an integer of 2 to 80. In a case in which the number of repetitions is from 2 to 100, the number of carbon atoms of the repeated alkylene oxide groups may be identical or may be different. In a case in which Y represents an alkyl group, the structure is not particularly limited; however, the alkyl group may be a linear group, a branched group, or a cyclic group. W represents an active ester group.

The “active ester group” used in this invention means an ester group which has an electron-withdrawing group with a high degree of acidity as a substituent of one side of the ester group, and is thereby activated for a nucleophilic reaction, that is, an ester group having high reaction activity. The active ester group is a term that is conventionally used in various fields of chemical synthesis, for example, polymer chemistry and peptide synthesis. On a practical level, phenolic esters, thiophenolic esters, N-hydroxyamine esters, esters of heterocyclic hydroxyl compounds, and the like are known as active ester groups having much higher activity compared to alkyl esters and the like.

Examples of such an active ester group include a p-nitrophenyl active ester group, a N-hydroxysuccinimide active ester group, a succinic acid imide active ester group, a phthalic acid imide active ester group, and a 5-norbornene-2,3-dicarboxyimide active ester group. A p-nitrophenyl active ester group or a N-hydroxysuccinimide active ester group is preferred, and a p-nitrophenyl active ester group is most preferred.

Subsequently, the constituent unit that is present in the copolymer (A) only will be described in detail.

It is preferable that the constituent unit derived from (c) an ethylenically unsaturated polymerizable monomer having one ethylenic double bond and a functional group that can be crosslinked to the substrate plate, is obtained by polymerizing an ethylenically unsaturated polymerizable monomer having a functional group capable of crosslinking.

The functional group capable of crosslinking is not particularly limited as long as the crosslinking reaction thereof does not proceed during the synthesis of a polymer compound, and for example, a functional group that produces a silanol group as a result of hydrolysis, an epoxy group, a (meth)acryl group, and a glycidyl group are used. However, from the viewpoint that the crosslinking treatment is easy, a functional group that produces a silanol group as a result of hydrolysis, an epoxy group, and a glycidyl group are preferred, and from the viewpoint that crosslinking can be achieved at lower temperature, a functional group that produces a silanol group as a result of hydrolysis is preferred.

A functional group that produces a silanol group as a result of hydrolysis is a group which is readily subjected to hydrolysis when brought into contact with water, and produces a silanol group. Examples thereof include a halogenated silyl group, an alkoxysilyl group, a phenoxysilyl group, and an acetoxysilyl group. Among them, from the viewpoint of not containing halogen atoms, an alkoxysilyl group, a phenoxysilyl group, and an acetoxysilyl group are preferred, and above all, from the viewpoint that a silanol group can be easily produced, an alkoxysilyl group is most preferred.

It is preferable that the ethylenically unsaturated polymerizable monomer having a functional group that produces a silanol group as a result of hydrolysis, is an ethylenically unsaturated polymerizable monomer represented by General Formula [3], in which a (meth)acryl group is bonded to the silicon atom to which at least one group capable of hydrolysis is bonded, either directly or via an alkyl chain having 1 to 20 carbon atoms.

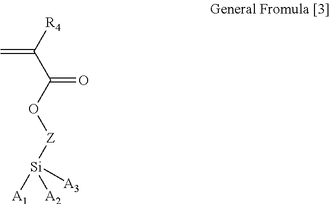

In General Formula [3], R₄ represents a hydrogen atom or a methyl group. Z represents an alkyl group having 1 to 20 carbon atoms, and the structure is not particularly limited. Therefore, the alkyl group may be a linear group, a branched group, or a cyclic group. At least one of A₁, A₂ and A₃ represents a group capable of hydrolysis, and is preferably any one of a methoxy group, an ethoxy group, a phenoxy group, and an acetoxy group. The others are inert groups that are not hydrolyzable, such as a methyl group or an ethyl group.

Examples of the ethylenically unsaturated polymerizable monomer in which a (meth)acryl group is bonded to the silicon atom to which at least one group capable of hydrolysis is bonded, either directly or through an alkyl chain having 1 to 20 carbon atoms, include (meth)acryloxyalkylsilane compounds such as 3-(meth)acryloxypropenyltrimethoxysilane, 3-(meth)acryloxypropylbis(trimethylsiloxy)methylsilane, 3-(meth)acryloxypropyldimethylmethoxysilane, 3-(meth)acryloxypropyldimethylethoxysilane, 3-(meth)acryloxypropylmethyldimethoxysilane, 3-(meth)acryloxypropylmethyldiethoxysilane, 3-(meth)acryloxypropyltrimethoxysilane, 3-(meth)acryloxypropyltriethoxysilane, 3-(meth)acryloxypropyltris(methoxyethoxy)silane, 8-(meth)acryloxyoctanyltrimethoxysilane, and 11-(meth)acryloxyundenyltrimethoxysilane. Among them, 3-methacryloxypropyltrimethoxysilane, 3-methacryloxypropyltriethoxysilane, 3-methacryloxypropyldimethylmethoxysilane, and 3-methacryloxypropyldimethylethoxysilane are preferred from the viewpoint of having excellent copolymerizability with an ethylenically unsaturated polymerizable monomer having an alkylene glycol residue, from the viewpoint of being easily available, and the like. These ethylenically unsaturated polymerizable monomers having an alkoxysilyl group are used singly, or in combination of two or more kinds thereof.

The copolymer (A) that is used for this invention may also include, in addition to the constituent unit derived from (a) an ethylenically unsaturated polymerizable monomer having one ethylenic double bond and an alkylene glycol residue, a constituent unit derived from (b) an ethylenically unsaturated polymerizable monomer having one ethylenic double bond and a functional group for immobilizing a physiologically active substance, and (c) a constituent unit derived from an ethylenically unsaturated polymerizable monomer having one ethylenic double bond and a functional group capable of crosslinking as described above, a constituent unit derived from (d) an ethylenically unsaturated polymerizable monomer represented by General Formula [6] having one ethylenic double bond and a hydrophobic group.

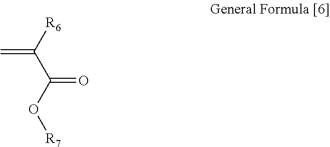

In General Formula [6], R₆ represents a hydrogen atom or a methyl group. R₇ represents a hydrophobic group, and although the structure is not particularly limited, an alkyl group or an aromatic group is preferred. More preferably, R₇ is an alkyl group having 1 to 20 carbon atoms. The structure of the alkyl group is not particularly limited, and the alkyl group may be a linear group, a branched group, or a cyclic group.

The (d) ethylenically unsaturated polymerizable monomer having one ethylenic double bond and a hydrophobic group is preferably n-butyl methacrylate, n-hexyl methacrylate, n-dodecyl methacrylate, n-octyl methacrylate, cyclohexyl methacrylate, or isobutyl methacrylate.

Next, the copolymer (A) will be explained. The copolymer (A) is obtainable by copolymerizing at least the (a) ethylenically unsaturated polymerizable monomer having one ethylenic double bond and an alkylene glycol residue, the (b) ethylenically unsaturated polymerizable monomer having one ethylenic double bond and a functional group for immobilizing a physiologically active substance, and the (c) ethylenically unsaturated polymerizable monomer having one ethylenic double bond and a functional group capable of crosslinking, as described previously. This polymer compound is a polymer that combinedly has a property of suppressing non-specific adsorption of a physiologically active substance, a property of immobilizing a physiologically active substance, and a property of crosslinking polymer main chains. In this polymer, the alkylene glycol residue accomplishes the role of suppressing non-specific adsorption of a physiologically active substance, and the functional group for immobilizing a physiologically active substance accomplishes the role of immobilizing a physiologically active substance.

The structure of the copolymer (A) is not particularly limited; however, a polymer compound represented by the following General Formula [4] can be suitably used.

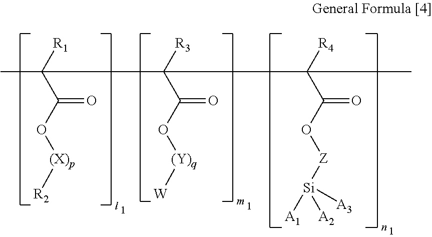

The compound of General Formula [4] is formed from three constituent units (repeating units) as illustrated in the FIGURE. The constituent unit shown on the left-hand side is hydrophilic, and therefore, the constituent unit accomplishes the role of suppressing non-specific adsorption of a protein or the like. The constituent unit shown in the middle has an active ester group, and therefore, the constituent unit accomplishes the role of immobilizing a physiologically active substance having an amino group. The constituent unit shown on the right-hand side forms a silanol when hydrolyzed, and therefore, the constituent unit accomplishes the role of preventing outflow at the time of washing through bonding to a solid-phase substrate or crosslinking between polymer molecules. In the following, the respective constituent units will be explained in detail.

In General Formula [4], in the constituent unit shown on the left-hand side, R₁ represents a hydrogen atom or a methyl group; and R₂ represents a hydrogen atom, a methyl group, or an ethyl group. X represents an alkylene glycol residue, and the number of carbon atoms thereof is 1 to 10, preferably 1 to 6, more preferably 2 to 4, even more preferably 2 or 3, and most preferably 2. The number of repetitions p of the alkylene glycol residue X is not particularly limited; however, the number of repetitions is preferably an integer of 1 to 100, more preferably an integer of 2 to 100, even more preferably an integer of 2 to 95, and most preferably an integer of 3 to 90. In a case in which the number of repetitions p is from 2 to 100, the numbers of carbon atoms of the repeated alkylene glycol residues X may be identical or may be different.

In General Formula [4], in regard to the constituent unit shown in the middle, R₃ represents a hydrogen atom or a methyl group. Y is preferably a chain of alkylene glycol residues each having 1 to 10 carbon atoms, or an alkyl group. In a case in which Y represents an alkylene glycol residue, the number of carbon atoms of Y is 1 to 10, preferably 1 to 6, more preferably 2 to 4, even more preferably 2 or 3, and most preferably 2. The number of repetitions q of the alkylene glycol residue Y is an integer of 1 to 100, more preferably an integer of 2 to 90, and most preferably an integer of 2 to 80. In a case in which the number of repetitions is from 2 to 100, the numbers of carbon atoms of the repeated alkylene glycol residues may be identical or may be different. In a case in which Y represents an alkyl group, the structure is not particularly limited; however, the alkyl group may be a linear group, a branched group, or a cyclic group. W represents an active ester group.

In General Formula [4], in regard to the constituent unit shown on the left-hand side, R₄ represents a hydrogen atom or a methyl group. Z represents an alkyl group having 1 to 20 carbon atoms, and the structure is not particularly limited. Therefore, the alkyl group may be a linear group, a branched group, or a cyclic group. At least one of A₁, A₂ and A₃ represents a group capable of hydrolysis, and is preferably any one of a methoxy group, an ethoxy group, a phenoxy group, and an acetoxy group. The others are inert groups that are not hydrolyzable, such as a methyl group or an ethyl group.

Furthermore, the copolymer (A) may further have another constituent unit, as shown in the following General Formula [7], in addition to the three constituent units described above.

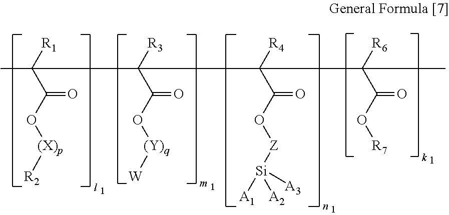

In General Formula [7], R₆ represents a hydrogen atom or a methyl group. R₇ represents a hydrophobic group, and although the structure is not particularly limited, an alkyl group or an aromatic group is preferred. R₇ is more preferably an alkyl group having 1 to 20 carbon atoms. The structure of the alkyl group is not particularly limited, and the alkyl group may be a linear group, a branched group, or a cyclic group.

The compositional proportion of the constituent unit having a hydrophilic group, which is included in the copolymer (A) of the invention (ratio of l₁ with respect to the sum of l₁, m₁, n₁, and k₁), is not particularly limited; however, the compositional proportion is preferably 5 to 98 mol %, more preferably 10 to 90 mol %, and most preferably 10 to 80 mol %, with respect to all of the constituent units of the polymer compound. When the compositional ratio is more than or equal to the lower limit, non-specific adsorption tends to be suppressed. On the other hand, when the compositional ratio is less than or equal to the upper limit, since the proportions of the other components become relatively larger, there is a tendency that the signal is increased, and outflow of the copolymer at the time of washing can be suppressed.

The compositional proportion of the constituent unit having an active ester group, which is included in the copolymer (A) of the invention (ratio of m₁ with respect to the sum of l₁, m₁, n₁, and k₁), is not particularly limited; however, the compositional proportion is preferably 1 to 94 mol %, more preferably 2 to 90 mol %, and most preferably 3 to 80 mol %, with respect to all of the constituent units of polymer compound A. When the compositional ratio is more than or equal to the lower limit, there is a tendency that the biological substance can be sufficiently immobilized. On the other hand, when the compositional ratio is less than or equal to the upper limit, there is a tendency for non-specific adsorption to be suppressed.

The compositional proportion of the constituent unit that forms a silanol when hydrolyzed, which is included in the copolymer (A) of the invention (ratio of n₁ with respect to the sum of l₁, m₁, n₁, and k₁), is not particularly limited; however, the compositional proportion is preferably 0.01 to 30 mol %, more preferably 0.1 to 20 mol %, and most preferably 0.1 to 10 mol %, with respect to all of the constituent units of the copolymer (A). When the compositional ratio is more than or equal to the lower limit, there is a tendency that the copolymer can be sufficiently immobilized on a solid-phase substrate. On the other hand, when the compositional ratio is less than or equal to the upper limit, there is a tendency for non-specific adsorption to be suppressed.

The compositional proportion of the constituent unit having a hydrophobic group, which is included in the copolymer (A) of the invention (ratio of k₁ with respect to the sum of l₁, m₁, n₁, and k₁), is not particularly limited; however, the compositional proportion is preferably 0 to 80 mol %, more preferably 0 to 70 mol %, and most preferably 0 to 50 mol %, with respect to all of the constituent units of the copolymer (A). When the compositional proportion is less than or equal to the upper limit, there is a tendency for non-specific adsorption to be suppressed.

Regarding the chemical structure of the copolymer (A) of the invention, as long as the chemical structure is a structure including at least a constituent unit having a hydrophilic group, a constituent unit having an active ester group, and a constituent unit that forms a silanol when hydrolyzed, the mode of linkage may be in any of a random form, a block form, or a graft form.

Next, the copolymer (B), the other copolymer, will be explained. The copolymer (B) is a polymer compound obtainable by copolymerizing at least (a) an ethylenically unsaturated polymerizable monomer having one ethylenic double bond and an alkylene glycol residue, and (b) an ethylenically unsaturated polymerizable monomer having one ethylenic double bond and a functional group for immobilizing a physiologically active substance, as described previously, the polymer compound having a reactive functional group at the terminal of at least one side of the compound.

This copolymer (B) is a polymer combinedly having a property of suppressing non-specific adsorption of a physiologically active substance and a property of immobilizing a physiologically active substance, in which the alkylene glycol residue accomplishes the role of suppressing non-specific adsorption of a physiologically active substance, and the functional group for immobilizing a physiologically active substance accomplishes the role of immobilizing a physiologically active substance. Furthermore, the copolymer (B) can be chemically bonded to a solid-phase substrate or the copolymer (A) by means of the terminal reactive functional group.

Specifically, regarding the copolymer (B), a polymer compound represented by the following General Formula [5] can be suitably used.

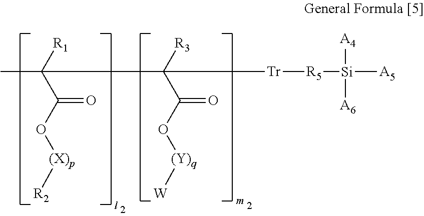

The compound of General Formula [5] is a compound in which, as shown in the FIGURE, two constituent units (repeating units) and a terminal silane compound are linked via a group derived from a chain transfer agent represented by Tr and a linker represented by R₅. Between the two constituent units, the constituent unit shown on the left-hand side is hydrophilic, and thus accomplishes the role of suppressing non-specific adsorption of a protein or the like. The constituent unit shown on the right-hand side has an active ester group, and thus accomplishes the role of immobilizing a physiologically active substance having an amino group. Furthermore, the terminal silane compound is able to form a silanol when hydrolyzed, and thus accomplishes the role of being bonded to a solid-phase substrate or another polymer and preventing outflow at the time of washing. In the following, the respective constituent units will be explained in detail.

In General Formula [5], in regard to the constituent unit shown on the left-hand side, R₁ represents a hydrogen atom or a methyl group; and R₂ represents a hydrogen atom, a methyl group, or an ethyl group. X represents an alkylene glycol residue, and the number of carbon atoms thereof is 1 to 10, preferably 1 to 6, more preferably 2 to 4, even more preferably 2 or 3, and most preferably 2. The number of repetitions p of the alkylene glycol residue X is not particularly limited; however, the number of repetitions is preferably an integer of 1 to 100, more preferably an integer of 2 to 100, even more preferably an integer of 2 to 95, and most preferably an integer of 3 to 90. In a case in which the number of repetitions p is from 2 to 100, the numbers of carbon atoms of the repeated alkylene glycol residues X may be identical or may be different.

In General Formula [5], in regard to the constituent unit shown on the right-hand side, R₃ represents a hydrogen atom or a methyl group. Y is preferably a chain of alkylene glycol residues each having 1 to 10 carbon atoms, or an alkyl group. In a case in which Y represents an alkylene glycol residue, the number of carbon atoms of Y is 1 to 10, preferably 1 to 6, more preferably 2 to 4, even more preferably 2 or 3, and most preferably 2. The number of repetitions q of the alkylene glycol residue Y is an integer of 1 to 100, more preferably an integer of 2 to 90, and most preferably an integer of 2 to 80. In a case in which the number of repetitions is from 2 to 100, the numbers of carbon atoms of the repeated alkylene glycol residues may be identical or may be different. In a case in which Y represents an alkyl group, the structure is not particularly limited; however, the alkyl group may be a linear group, a branched group, or a cyclic group. W represents an active ester group.

In General Formula [5], in regard to the silane compound and the other parts, at least one of A₄, A₅, and A₆ represents a group capable of hydrolysis, and is preferably any one of a methoxy group, an ethoxy group, a phenoxy group, and an acetoxy group. The others are inert groups that are not hydrolyzable, such as a methyl group or an ethyl group. R₅ is not particularly limited; however, a hydrocarbon chain having 1 to 20 carbon atoms, which may be interrupted by —O—, —S—, —NH—, —CO—, or —CONH—, is preferred. The structure of the hydrocarbon chain is not particularly limited; however, the hydrocarbon chain may be a linear group, a branched group, or a cyclic group. Tr represents a group derived from a chain transfer agent. There are no particular limitations on the chain transfer agent; however, it is preferable that the chain transfer agent has a mercapto group.

Furthermore, the copolymer (B) may further have another constituent unit, as shown in the following General Formula [8], in addition to the two constituent units and the silane compound described above.

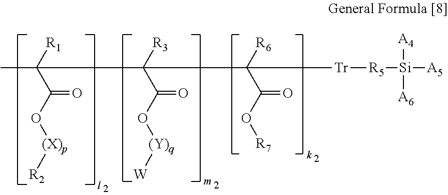

In General Formula [8], R₆ represents a hydrogen atom or a methyl group. R₇ represents a hydrophobic group, and although the structure is not particularly limited, R₇ is preferably an alkyl group or an aromatic group. More preferably, R₇ is an alkyl group having 1 to 20 carbon atoms. The structure of the alkyl group is not particularly limited, and the alkyl group may be a linear group, a branched group, or a cyclic group.

The compositional proportion of the constituent unit having a hydrophilic group, which is included in copolymer (B) of the invention (ratio of l₂ with respect to the sum of l₂, m₂, and k₂), is not particularly limited; however, the compositional proportion is preferably 5 to 98 mol %, more preferably 10 to 90 mol %, and most preferably 10 to 80 mol %, with respect to all of the constituent units of the copolymer (B). When the compositional ratio is more than or equal to the lower limit, there is a tendency that non-specific adsorption can be suppressed. On the other hand, when the compositional ratio is less than or equal to the upper limit, since the proportions of the other components become relatively large, there is a tendency that the signal is increased, and outflow of the copolymer at the time of washing can be suppressed.

The compositional proportion of the constituent unit having an active ester group, which is included in the copolymer (B) of the invention (ratio of m₂ with respect to the sum of l₂, m₂, and k₂), is not particularly limited; however, the compositional proportion is preferably 1 to 94 mol %, more preferably 2 to 90 mol %, and most preferably 3 to 80 mol %, with respect to all of the constituent units of the copolymer (B). When the compositional ratio is more than or equal to the lower limit, there is a tendency that a biological substance can be sufficiently immobilized. On the other hand, when the compositional ratio is less than or equal to the upper limit, there is a tendency for non-specific adsorption to be suppressed.

The compositional proportion of the constituent unit having a hydrophobic group, which is included in the copolymer (B) of the invention (ratio of k₂ with respect to the sum of l₂, m₂, and k₂), is not particularly limited; however, the compositional proportion is preferably 0 to 80 mol %, more preferably 0 to 70 mol %, and most preferably 0 to 50 mol %, with respect to all of the constituent units of the copolymer (B). When the compositional proportion is less than or equal to the upper limit, there is a tendency for non-specific adsorption to be suppressed.

Regarding the chemical structure of the copolymer (B) of the invention, as long as the chemical structure is a structure having at least a constituent unit having a hydrophilic group and a constituent unit having an active ester group, and has a silane compound that forms a silanol when hydrolyzed, at the terminal, the mode of linkage may be in any of a random form, a block form, or a graft form.

The method for polymerizing the copolymer (A) of the invention is not particularly limited; however, from the viewpoint of ease of synthesis, it is preferable to subject a mixture including at least (a) an ethylenically unsaturated polymerizable monomer having one ethylenic double bond and an alkylene glycol residue, (b) an ethylenically unsaturated polymerizable monomer having one ethylenic double bond and a functional group for immobilizing a physiologically active substance, and (c) an ethylenically unsaturated polymerizable monomer having one ethylenic double bond and a functional group capable of crosslinking, to radical polymerization in a solvent in the presence of a polymerization initiator.

Regarding the reaction solvent, any solvent capable of dissolving the respective ethylenically unsaturated polymerizable monomers may be used, and examples thereof include methanol, ethanol, t-butyl alcohol, benzene, toluene, tetrahydrofuran, dioxane, dichloromethane, chloroform, and methyl ethyl ketone. These solvents are used singly or in combination of two or more kinds thereof. In a case in which the polymer compounds are applied on a plastic substrate, ethanol, methanol, and methyl ethyl ketone are preferred because these solvents do not modify the substrate.

Regarding the copolymer (A) of the invention, the copolymer can be obtained by performing polymerization in the co-presence of the various monomers.

The polymerization initiator may be any conventional radical initiator, and examples thereof include azo compounds such as 2,2′-azobisisobutyronitrile (hereinafter, referred to as “AIBN”) and 1,1′-azobis(cyclohexane-1-carbonitrile); and organic peroxides such as benzoyl peroxide and lauryl peroxide.

The method for polymerizing the copolymer (B) of the invention is not particularly limited; however, from the viewpoint of ease of synthesis, it is preferable to subject a mixture including at least (a) an ethylenically unsaturated polymerizable monomer having one ethylenic double bond and an alkylene glycol residue, (b) an ethylenically unsaturated polymerizable monomer having one ethylenic double bond and a functional group for immobilizing a physiologically active substance, and a silane coupling agent having a chain transfer group and capable of producing a silanol as a result of hydrolysis, to radical polymerization in a solvent in the presence of a polymerization initiator. The specific synthesis method is the same as the method for the copolymer (A).

When the coating agent composition of the present embodiment is used, a property of immobilizing a particular physiologically active substance can be easily imparted by coating the surface of a solid-phase substrate with copolymers (A) and (B) described above. Furthermore, since an alkylene glycol residue is present in the components of the copolymers, a property of suppressing non-specific adsorption of a physiologically active substance can be further imparted, in addition to the property of immobilizing a particular physiologically active substance. Furthermore, since the copolymer (A) combinedly has a property of being bonded to a substrate and a property of crosslinking polymer main chains, the copolymer molecules can be crosslinked after the substrate surface is coated. Thereby, insolubility can be imparted to the polymers on the substrate, and a decrease in the signal caused by substrate washing can be reduced. In regard to the copolymer (B), the terminal reactive group can be bonded to the substrate and the copolymer (A), the relevant copolymer can be chemically grafted. Therefore, there is no risk of a decrease in signal caused by substrate washing.

[Solid-Phase Substrate Having Coated Surface and Method for Producing Same (1)]

According to an embodiment of the invention, there is provided a method for producing a solid-phase substrate having a coated surface. The method for producing a solid-phase substrate related to the present embodiment includes steps of (i) preparing a copolymer solution in which the copolymers (A) and (B) described above are dissolved in a solvent (organic solvent) at a concentration of 0.05% by weight to 10% by weight; (ii) applying the copolymer solution on the surface of a solid-phase substrate by a known method such as immersion or spraying; and then (iii) drying the applied solution at room temperature or under heating. Meanwhile, according to the present specification, “drying” means removal of the solvent.

Thereafter, the main chains of the copolymers may be crosslinked by any arbitrary method in accordance with the functional group capable of crosslinking. In regard to coating of the copolymers in a case in which the functional group capable of crosslinking is a functional group that produces a silanol group as a result of hydrolysis, a mixed solution prepared by incorporating water into an organic solvent may also be used. The incorporated water induces hydrolysis, and silanol groups are produced in the copolymer. Furthermore, when the synthesized copolymers are heated, the main chains are bonded, and the copolymers become insoluble. When the ease of preparation of the solution is considered, a solvent having a water content of about 0.01% by weight to 15% by weight is preferred.

Regarding the solvent (organic solvent) for the copolymers, single solvents of ethanol, methanol, t-butyl alcohol, benzene, toluene, tetrahydrofuran, dioxane, dichloromethane, chloroform, acetone, methyl ethyl ketone, and the like, or mixed solvents thereof are used. Among them, ethanol, methanol, and methyl ethyl ketone are preferred because these solvents do not modify plastic substrates and can be easily dried. Furthermore, ethanol, methanol, and methyl ethyl ketone are preferred because these are miscible with water at any arbitrary proportions, even in a case in which a polymer compound is hydrolyzed in a solution.

In regard to coating of the substrate surface with the copolymers, in a case in which the copolymers (A) and (B) are used in mixture, the proportions of the copolymers are not particularly limited; however, it is preferable that the copolymer (A) is included at a proportion of 1% to 99%, more preferably 5% to 95%, and most preferably 10% to 90%, with respect to the total amount.

In regard to the process of applying a solution prepared by dissolving the copolymers of the invention on the substrate surface and then drying the solution, the silanol groups in the copolymer undergo dehydration condensation with silanol groups, hydroxyl groups, amino groups and the like in the other copolymer molecules, and form crosslinks. Furthermore, even in a case in which hydroxyl groups, carbonyl groups, amino groups, and the like are present on the substrate surface, these groups can be subjected to dehydration condensation as such and can be chemically bonded to the substrate surface. Since the covalent bonds formed by dehydration and condensation of silanol groups have a property of being not easily hydrolyzable, the copolymers applied on the substrate surface have no chance of being easily dissolved or being liberated from the substrate. Dehydration condensation of silanol groups is accelerated by a heating treatment. Therefore, a heating treatment may be performed in a temperature range in which the copolymers are not modified by heat, for example, at 60° C. to 120° C., for 5 minutes to 100 hours.

Furthermore, according to still another embodiment of the invention, there is provided a solid-phase substrate having a coated surface, which is obtained by the relevant production method.

(Solid-Phase Substrate)

Regarding the material for the substrate plate (solid-phase substrate) for a biosensor used for the invention, glass, plastics, metals, and others can be used. However, from the viewpoints of ease of the surface treatment and mass productivity, a plastic is preferred, and above all, a thermoplastic resin is more preferred. Furthermore, the solid-phase substrate may be in the form of for example, a plate, a film, or beads.

Regarding the thermoplastic resin, a resin having a smaller amount of fluorescence generation is preferred, and for example, it is preferable to use a straight chain-like polyolefin such as polyethylene or polypropylene; a cyclic polyolefin; or a fluororesin. It is more preferable to use a saturated cyclic polyolefin, which has particularly excellent heat resistance, chemical resistance and moldability, with less fluorescence. Here, a saturated cyclic polyolefin refers to a saturated polymer obtainable by hydrogenating a polymer having a cyclic olefin structure alone, or by hydrogenating a copolymer of a cyclic olefin and an α-olefin.

In order to increase the adhesiveness between the solid-phase substrate surface and the copolymers applied on the surface, or to graft the copolymers to the solid-phase substrate, it is preferable to activate the surface of the solid-phase substrate. Regarding the means for activating the surface, a method of performing a plasma treatment under the conditions of an oxygen atmosphere, an argon atmosphere, a nitrogen atmosphere, or an air atmosphere; a method of treating the surface with an excimer laser such as ArF or

KrF; and the like may be used, and a method of performing a plasma treatment in an oxygen atmosphere is preferred.

A substrate plate for a biosensor (solid-phase substrate), which has excellent immobilization ability for physiologically active substances and in which non-specific adsorption of physiologically active substances to the substrate is suppressed, can be easily produced by applying the copolymers of the invention on a solid-phase substrate. Furthermore, insolubility can be imparted to the copolymers on the substrate by crosslinking the copolymers. Furthermore, since the copolymers can be bonded to the substrate plate by chemical bonding, there is no outflow in the washing process. From these points of view, a substrate coated with the copolymers can be suitably used for a biosensor.

[Solid-Phase Substrate Having Coated Surface and Method for Producing Same (2)]

According to still another embodiment of the invention, there is provided a method for producing a solid-phase substrate having a coated surface, the method including a step of applying a first coating agent composition including the copolymer (A) described above and a solvent on the surface of a solid-phase substrate; a step of removing the solvent from the first coating agent composition applied on the solid-phase substrate, and obtaining a solid-phase substrate having a surface coated with the copolymer (A); a step of applying a second coating agent composition including the copolymer (B) described above and a solvent on the surface of the solid-phase substrate having a surface coated with the copolymer (A); and a step of removing the solvent from the second coating agent composition applied on the solid-phase substrate.

Furthermore, according to still another embodiment of the invention, there is provided a solid-phase substrate having a coated surface, which is obtainable by the relevant production method.

In the method for producing a solid-phase substrate of the present embodiment, regarding the solvent for the copolymers, solvents similar to those mentioned above can be used.

[Coating Agent Kit]

According to still another embodiment of the invention, there is provided a coating agent kit comprising the copolymer (A) and the copolymer (B) described above, respectively accommodated in different containers, which is used to coat the surface of a solid-phase substrate.

When the kit of the present embodiment is used, a solid-phase substrate having a coated surface may be produced using a composition prepared by mixing the copolymer (A) and the copolymer (B). A solid-phase substrate having a coated surface may also be produced by first coating a solid-phase substrate with the copolymer (A), and subsequently coating the solid-phase substrate with the copolymer (B).

As will be described below in Examples, a solid-phase substrate having excellent immobilization ability for physiologically active substances and exhibiting less non-specific adsorption can be produced by any of the methods described above.

[Biosensor and Method for Producing Same]

According to still another embodiment of the invention, there is provided a biosensor in which a physiologically active substance is immobilized on the solid-phase substrate having a coated surface as described above. Furthermore, according to still another embodiment of the invention, there is provided a method for producing a biosensor, the method including a step of immobilizing a physiologically active substance on the solid-phase substrate having a coated surface described above.

Various physiologically active substances can be immobilized using the solid-phase substrate having a coated surface (substrate plate for a biosensor) as described above. Examples of the physiologically active substance to be immobilized include a nucleic acid, an aptamer, a protein, an oligopeptide, a sugar chain, and a glycoprotein. For example, in a case in which a sugar chain is immobilized, it is preferable to introduce an amino group in order to increase reactivity with the active ester group. The position of introduction of the amino group may be at a terminal of the molecular chain or in a side chain (also called “branch”); however, it is preferable that the amino group is introduced at a terminal of the molecular chain.

When a physiologically active substance is immobilized on a substrate plate for a biosensor in the present invention, a method of spot-applying (spotting) a liquid having a physiologically active substance dissolved or dispersed therein is preferred.

After the spot application, when the substrate plate is left to stand still, the physiologically active substance is immobilized on the surface. For example, in a case in which an aminated sugar chain is used, immobilization can be achieved by leaving the substrate plate to stand at a temperature ranging from room temperature to 80° C. for 1 hour to 4 hours. A higher treatment temperature is more preferred. The liquid for dissolving or dispersing the physiologically active substance is preferably a weakly alkaline liquid.

After washing, the functional groups on the substrate plate surface, excluding the part on which the physiologically active substance is immobilized, are subjected to inactivation. In the case of an active ester or an aldehyde group, it is preferable to perform the inactivation using an alkali compound or a compound having a primary amino group.

Regarding the alkali compound, sodium hydroxide, potassium hydroxide, sodium carbonate, sodium hydrogen carbonate, disodium hydrogen phosphate, calcium hydroxide, magnesium hydroxide, sodium borate, lithium hydroxide, potassium phosphate, and the like can be preferably used.

Regarding the compound having a primary amino group, methylamine, ethylamine, butylamine, glycine, 9-aminoaquadine, aminobutanol, 4-aminobutyric acid, aminocaprylic acid, aminoethanol, 5-amino-2,3-dihydro-1,4-pentanol, aminoethanethiol hydrochloride, aminoethanethiol sulfuric acid, 2-(2-aminoethylamino)ethanol, 2-aminoethyl dihydrogen phosphate, aminoethyl hydrogen sulfate, 4-(2-aminoethyl)morpholine, 5-aminofluorescein, 6-aminohexanoic acid, aminohexyl cellulose, p-aminohippuric acid, 2-amino-2-hydroxymethyl-1,3-propanediol, 5-aminoisophthalic acid, aminomethane, aminophenol, 2-aminooctane, 2-aminooctanoic acid, 1-amino-2-propanol, 3-amino-1-propanol, 3-aminopropene, 3-aminopropionitrile, aminopyridine, 11-aminoundecanoic acid, aminosalicylic acid, aminoquinoline, 4-aminophthalonitrile, 3-aminophthalimide, p-aminopropiophenone, aminophenylacetic acid, aminonaphthalene, and the like can be preferably used. Aminoethanol and glycine are most preferred.

A biosensor obtained by immobilizing a physiologically active substance as such can be used for many analytic systems including immunodiagnostic systems, gene microarray systems, protein microarray systems, sugar chain microarray systems, and microfluidics devices.

### EXAMPLES

Hereinafter, the invention will be explained more specifically by way of Examples, but the technical scope of this invention is not intended to be limited to these Examples.

### Synthesis of p-Nitrophenyloxycarbonyl-Polyethylene Glycol Methacrylate (MEONP)

0.01 mol of polyethylene glycol monomethacrylate (BLENMER PE-200 (n=4), manufactured by NOF Corporation) was dissolved in 20 mL of chloroform, and then the solution was cooled to −30° C. While the solution was maintained at −30° C., a uniform solution of 0.01 mol of p-nitrophenyl chloroformate (manufactured by Sigma-Aldrich Company), 0.01 mol of triethylamine (manufactured by Wako Pure Chemical Industries, Ltd.), and 20 mL of chloroform, which had been prepared in advance, was slowly added dropwise to the solution. The mixture was allowed to react for 1 hour at −30° C., and then the solution was stirred for 2 hours. Subsequently, salts were removed by filtration from the reaction liquid, the solvent was distilled off, and thus p-nitrophenyloxycarbonyl-polyethylene glycol methacrylate (MEONP) was obtained. The monomer thus obtained was analyzed by ¹H-NMR in deuterated chloroform solvent, and it was confirmed that the monomer contained 4.5 units of ethylene glycol residue.

### Synthesis of Copolymer (A)

### Synthesis Example (A-1)

Polyethylene glycol methyl ether methacrylate (also known as methoxypolyethylene glycol methacrylate) (PEGMA, number average molecular weight Mn=468, manufactured by Shin Nakamura Chemical Co., Ltd.), MEONP, and 3-methacryloxypropyldimethylethoxysilane (MPDES, manufactured by Gelest, Inc.) were dissolved in this order in dehydrated ethanol at concentrations of 0.90 mol/L, 0.05 mol/L, and 0.05 mol/L, respectively. Thus, a mixed monomer solution was produced. 2,2-azobisisobutyronitrile (AIBN, manufactured by Wako Pure Chemical Industries, Ltd.) at 0.002 mol/L was further added thereto, and the mixed monomer solution was stirred until the solution became uniform. Subsequently, the mixed monomer solution was allowed to react for 4 hours at 60° C. in an argon gas atmosphere, subsequently the reaction solution was added dropwise to diethyl ether, and a precipitate was collected. A copolymer (A-1) thus obtained was analyzed by ¹H-NMR in deuterated chloroform solvent, and the compositional ratio of this copolymer (A-1) was calculated from the respective integral values of a peak originating from the methylene bonded to Si of MPDES, which appeared at near 0.7 ppm; a peak originating from the terminal methoxy group of PEGMA, which appeared at near 3.4 ppm; and peaks originating from the benzene ring of MEONP, which appeared at near 7.4 ppm and 8.3 ppm. The results are presented in Table 1.

### Synthesis of Copolymer (B)

### Synthesis Example (B-1)

A mixed monomer solution was produced by dissolving polyethylene glycol methyl ether methacrylate (also known as methoxypolyethylene glycol methacrylate) (PEGMA, number average molecular weight Mn=468, manufactured by Shin Nakamura Chemical Co., Ltd.) and MEONP in 2-butanone. The total monomer concentration was 0.7 mol/L, and the molar ratio of PEGMA and MEONP in this order was 50:50. To this solution, (3-mercaptopropyl)dimethylmethoxysilane (hereinafter, described as MPDMS, manufactured by Sigma-Aldrich Company) was added to a concentration of 0.015 mol/L, and 2,2-azobisisobutyronitrile (hereinafter, described as AIBN, manufactured by Wako Pure Chemical Industries, Ltd.) was added to a concentration of 0.05 mol/L. The mixture was stirred until it became uniform. Subsequently, the mixture was allowed to react for 24 hours at 60° C. in an argon gas atmosphere, and then the reaction solution was added dropwise to a mixed solvent of hexane and acetone (mixing ratio was 4:1 as a volume ratio). A precipitate was collected. A copolymer (B-1) thus obtained was analyzed by ¹H-NMR in deuterated chloroform solvent, and the compositional ratio of this copolymer (B-1) was calculated from the respective integral values of a peak originating from the terminal methoxy group of PEGMA, which appeared at near 3.4 ppm; peaks originating from the benzene ring of MEONP, which appeared at near 7.6 ppm and 8.4 ppm; and a peak originating from the methylene bonded to Si of MPDMS, which appeared at near 0.7 ppm. The results are presented in Table 1.

### Synthesis Example (B-2)

A mixed monomer solution was produced by dissolving polyethylene glycol methyl ether methacrylate (also known as methoxypolyethylene glycol methacrylate) (PEGMA, number average molecular weight Mn=468, manufactured by Shin Nakamura Chemical Co., Ltd.), and MEONP in a mixed solvent of ethanol/2-butanone=9/1. The total monomer concentration was 0.7 mol/L, and the molar ratio of PEGMA and MEONP in this order was 80:20. To this solution, (3-mercaptopropyl)dimethylmethoxysilane (hereinafter, described as MPDMS, manufactured by Sigma-Aldrich Company) was added to a concentration of 0.015 mol/L, and 2,2-azobisisobutyronitrile (hereinafter, described as AIBN, manufactured by Wako Pure Chemical Industries, Ltd.) was added to a concentration of 0.02 mol/L. The mixture was stirred until it became uniform. Subsequently, the mixture was allowed to react for 24 hours at 60° C. in an argon gas atmosphere, and then the reaction solution was added dropwise to a mixed solvent of hexane and acetone (mixing ratio was 4:1 as a volume ratio). A precipitate was collected. Copolymer (B-2) thus obtained was analyzed by ¹H-NMR in deuteratated chloroform solvent, and the compositional ratio of this copolymer (B-2) was calculated from the respective integral values of a peak originating from the terminal methoxy group of PEGMA, which appeared at near 3.4 ppm; peaks originating from the benzene ring of MEONP, which appeared at near 7.6 ppm and 8.4 ppm; and a peak originating from the methylene bonded to Si of MPDMS, which appeared at near 0.7 ppm. The results are presented in Table 1.

TABLE 1

| | | Synthesis | Synthesis | Synthesis |
|---|---|---|---|---|
| | | Example | Example | Example |
| | | (A-1) | (B-1) | (B-2) |
| Compositional | PEGMA | 90 | 51 | 81 |
| ratio (mol %) | MEONP | 5 | 47 | 17 |
| determined by | MPDES | 4 | — | — |
| NMR | MPDMS | — | 2 | 2 |

### Example 1

A saturated cyclic polyolefin resin (hydrogenation product of a ring-opened polymer of 5-methyl-2-norbornene, Melt Flow Rate (MFR): 21 g/10 min, hydrogenation ratio: substantially 100%, thermal deformation temperature: 123° C.) was used and processed into a slide glass shape (dimension: 76 mm×26 mm×1 mm) by injection molding. Thus, a solid-phase substrate plate was produced. The substrate plate surface was subjected to an oxidation treatment by performing a plasma treatment in an oxygen atmosphere. This solid-phase substrate plate was immersed in a 0.3% by weight methyl ethyl ketone mixed solution of the polymer compounds obtained in Synthesis Example (A-1) for copolymer and Synthesis Example (B-1) for copolymer described above (ratio of copolymer (A-1) and copolymer (B-1) was 1:1), and thereby, a layer containing the two kinds of copolymers was introduced onto the substrate plate surface. This substrate plate was heated and dried for 72 hours at 100° C., and thereby, the substrate plate and the layer containing the polymers were chemically bonded. Thus, a substrate plate of Example 1 was obtained.

### Example 2

A saturated cyclic polyolefin resin (hydrogenation product of a ring-opened polymer of 5-methyl-2-norbornene, Melt Flow Rate (MFR): 21 g/10 min, hydrogenation ratio: substantially 100%, thermal deformation temperature: 123° C.) was used and processed into a slide glass shape (dimension: 76 mm×26 mm×1 mm) by injection molding. Thus, a solid-phase substrate plate was produced. The substrate plate surface was subjected to an oxidation treatment by performing a plasma treatment in an oxygen atmosphere. This solid-phase substrate plate was immersed in a 0.3% by weight methyl ethyl ketone mixed solution of the polymer compounds obtained in Synthesis Example (A-1) for copolymer described above, and thereby, a layer containing copolymer (A-1) only was introduced onto the substrate plate surface. This substrate plate was dried for 1 hour at room temperature. Subsequently, this solid-phase substrate plate was immersed in a 0.3% by weight methyl ethyl ketone mixed solution of Synthesis Example (B-1) for copolymer described above, and thereby, a layer containing copolymer (B-1) was introduced onto the substrate plate surface. This substrate plate was heated and dried for 72 hours at 100° C., and thereby, the substrate plate and the layer containing the polymers were chemically bonded. Thus, a substrate plate of Example 2 was obtained.

### Example 3

A saturated cyclic polyolefin resin (hydrogenation product of a ring-opened polymer of 5-methyl-2-norbornene, Melt Flow Rate (MFR): 21 g/10 min, hydrogenation ratio: substantially 100%, thermal deformation temperature: 123° C.) was used and processed into a slide glass shape (dimension: 76 mm×26 mm×1 mm) by injection molding. Thus, a solid-phase substrate plate was produced. The substrate plate surface was subjected to an oxidation treatment by performing a plasma treatment in an oxygen atmosphere. This solid-phase substrate plate was immersed in a 0.3% by weight methyl ethyl ketone mixed solution of Synthesis Example (A-1) for copolymer described above, and thereby, a layer containing copolymer (A-1) only was introduced onto the substrate plate surface. This substrate plate was dried for 1 hour at room temperature. Subsequently, this solid-phase substrate plate was immersed in a 0.3% by weight methyl ethyl ketone mixed solution of Synthesis Example (B-2) for copolymer described above, and thereby, a layer containing copolymer (B-2) was introduced onto the substrate plate surface. This substrate plate was heated and dried for 72 hours at 100° C., and thereby, the substrate plate and the layer containing the polymers were chemically bonded. Thus, a substrate plate of Example 3 was obtained.

### Comparative Example 1

### Aldehyde Substrate Plate

A saturated cyclic polyolefin resin (hydrogenation product of a ring-opened polymer of 5-methyl-2-norbornene, Melt Flow Rate (MFR): 21 g/10 min, hydrogenation ratio: substantially 100%, thermal deformation temperature: 123° C.) was used and processed into a slide glass shape (dimension: 76 mm×26 mm×1 mm) by injection molding. Thus, a solid-phase substrate plate was produced. The substrate plate surface was subjected to an oxidation treatment by performing a plasma treatment in an oxygen atmosphere. This substrate plate was immersed in a 2% by volume ethanol solution of 3-aminopropyltrimethoxysilane, and then was washed with pure water, followed by a heat treatment for 2 hours at 45° C. Thus, amino groups were introduced into the substrate plate. Furthermore, the substrate plate was further immersed in a 1% by volume aqueous solution of glutaraldehyde, and then was washed with pure water. Thus, aldehyde groups were introduced onto the substrate, and a substrate plate of Comparative Example 1 was obtained.

### Comparative Example 2

A saturated cyclic polyolefin resin (hydrogenation product of a ring-opened polymer of 5-methyl-2-norbornene, Melt Flow Rate (MFR): 21 g/10 min, hydrogenation ratio: substantially 100%, thermal deformation temperature: 123° C.) was used and processed into a slide glass shape (dimension: 76 mm×26 mm×1 mm) by injection molding. Thus, a solid-phase substrate plate was produced. The substrate plate surface was subjected to an oxidation treatment by performing a plasma treatment in an oxygen atmosphere. This solid-state substrate plate was immersed in a 0.3% by weight ethanol mixed solution of the polymer compound obtained in Synthesis Example (A-1) for copolymer described above, and thereby, a layer containing copolymer (A-1) only was introduced onto the substrate plate surface. This substrate plate was heated and dried for 72 hours at 100° C., and thereby, the substrate plate and the layer containing the polymer were chemically bonded. Thus, a substrate plate of Comparative Example 2 was obtained.

### Comparative Example 3

A saturated cyclic polyolefin resin (hydrogenation product of a ring-opened polymer of 5-methyl-2-norbornene, Melt Flow Rate (MFR): 21 g/10 min, hydrogenation ratio: substantially 100%, thermal deformation temperature: 123° C.) was used and processed into a slide glass shape (dimension: 76 mm×26 mm×1 mm) by injection molding. Thus, a solid-phase substrate plate was produced. The substrate plate surface was subjected to an oxidation treatment by performing a plasma treatment in an oxygen atmosphere. This solid-phase substrate plate was immersed in a 0.3% by weight methyl ethyl ketone mixed solution of the polymer compound obtained in Synthesis Example (B-1) for copolymer described above, and thereby, a layer containing copolymer (B-1) only was introduced onto the substrate plate surface. This substrate plate was heated and dried for 72 hours at 100° C., and thereby, the substrate plate and the layer containing the polymer were chemically bonded. Thus, a substrate plate of Comparative Example 3 was obtained.

### Evaluation Example 1

The following evaluations were performed for the respective substrate plates of Examples 1 to 3 and Comparative Examples 1 to 3.

Process 1 (Immobilization of Aminated Sugar Chain)

A sugar chain in which the reduced terminals had been aminated was diluted with a phosphate buffer (pH 8.5) at 0.3 mol/L to obtain a concentration of 200 mmol/L. This dilution was spotted on each of the substrate plates obtained in Examples and Comparative Examples, using an automated spotter, and then the substrate plates were left to stand for 1 hour in an environment at room temperature. Thereby, the aminated sugar chain was immobilized.

Process 2 (Adsorption Prevention Treatment)

Thereafter, the respective substrate plates of Examples and Comparative Examples 2 and 3 were immersed for 1 hour in an aqueous solution (pH 9.5) including ethanolamine (manufactured by Wako Pure Chemical Industries, Ltd., special grade) at 0.1 mol/L and Tris buffer (manufactured by Sigma-Aldrich Company) at 0.1 mol/L, and thereby, the remaining active ester parts were deactivated.

Furthermore, the substrate plate of Comparative Example 1 was subjected to an adsorption prevention treatment by immersing the substrate plate for 2 hours in a solution obtained by diluting a commercially available adsorption inhibitor, BLOCK ACE (manufactured by Dainippon Pharma Co., Ltd.) four times using a PBS buffer (manufactured by Nissui Pharmaceutical Co., Ltd., buffer obtained by dissolving Dulbecco's PBS (−) for culture at a composition of 9.6 g in 1 L of pure water) as a diluent.

Process 3 (Reaction with Serum)

Bovine serum albumin (hereinafter, described as BSA, manufactured by Sigma-Aldrich Company) was dissolved in a PBS buffer to a concentration of 3%, and polyoxyethylene (20) sorbitan monolaurate (manufactured by Wako Pure Chemical Industries, Ltd., corresponding to TWEEN 20; hereinafter, described as TWEEN 20 for convenience) was diluted in the solution to obtain a concentration of 1%. This solution was used to dilute a commercially available serum (manufactured by Gemini Bio-Products, Inc.) such that the incorporated IgG antibody concentration would be 1 mg/mL. This diluted serum solution was brought into contact with a substrate plate obtained in Process 2 for 90 minutes at 37° C., and thereby, a reaction between the antibodies in the serum and the sugar chains was induced. After the reaction, the substrate plate was washed once with a 0.1% TWEEN 20-containing PBS, and three times with a 0.001% TWEEN 20-containing PBS.

Process 4 (Reaction with Biotin-Labeled Antibodies)

BSA was dissolved in a PBS buffer to a concentration of 3%, and TWEEN 20 was diluted in this solution to a concentration of 0.1%. This solution was used to dilute a biotin-labeled anti-IgG antibody (manufactured by Thermo Fischer Scientific, Inc.) to a concentration of 10 μg/mL. This diluted solution was brought into contact with a substrate plate obtained in Process 3 for 45 minutes at 25° C., and thereby, a reaction between the antibody in the serum and the substrate plate was induced. After the reaction, the substrate plate was washed once with a 0.1% TWEEN 20-containing PBS, and three times with a 0.001% TWEEN 20-containing PBS.

Process 5 (Fluorescent Labeling Reaction)

Cy5-streptavidin (manufactured by GE Healthcare Corporation) was diluted with a 0.1% TWEEN 20-containing PBS to a concentration of 2 μg/mL. This diluted solution was brought into contact with a substrate plate obtained in Process 4 for 30 minutes at 25° C., and thereby, a fluorescent labeling reaction was induced. After the reaction, the substrate plate was washed once with a 0.1% TWEEN 20-containing PBS, three times with a 0.001% TWEEN 20-containing PBS, and once with pure water. The substrate plate was dried by centrifugation using a centrifuge.

An analysis of the amount of fluorescence was performed for each of the substrate plates, and the spot signal intensity value and the background value were evaluated. The results of the background value, the spot signal intensity, and the S/N ratio calculated from these values are presented in Table 2. For the measurement of the amount of fluorescence in Examples and Comparative Examples, a biochip scanner manufactured by PerkinElmer, Inc., “SCAN ARRAY”, was used. The conditions for measurement were as follows: laser output power: 90%, PMT sensitivity: 50%, excitation wavelength: 633 nm, measurement wavelength: 670 nm, and resolution: 10 μm.

From a comparison between Examples 1 to 3 and Comparative Examples 1 to 3, it can be said that the biochip substrate plate according to the invention is a biochip having a high signal intensity, with the background noise suppressed to a low level, and having an excellent S/N ratio.

TABLE 2

| | Signal intensity | Background | S/N ratio |
|---|---|---|---|
| Example 1 | 18747 | 87 | 215 |
| Example 2 | 46623 | 91 | 512 |
| Example 3 | 37581 | 93 | 404 |
| Comparative | 4560 | 506 | 9 |
| Example 1 | | | |
| Comparative | 2729 | 85 | 32 |
| Example 2 | | | |
| Comparative | 13581 | 85 | 160 |
| Example 3 | | | |

From a comparison between Examples 1 to 3 and Comparative Examples 1 to 3, it can be said that the biochip substrate plate according to the invention is a biochip having a high signal intensity, with the background noise suppressed to a low level, and having an excellent S/N ratio. Furthermore, in a case in which the copolymer (B) was applied after the copolymer (A) was applied on the substrate plate, the signal intensity of the biochip substrate plate was further increased, compared to the case in which a mixture of the copolymers (A) and (B) was applied on the substrate plate.

### INDUSTRIAL APPLICABILITY

According to the invention, a biodevice having a high S/N ratio can be provided.

1: A coating agent composition for coating a surface of a solid-phase substrate, comprising:
a first copolymer having a first repeating unit, a second repeating unit, and a third repeating unit; and
a second copolymer having a fourth repeating unit and a fifth repeating unit, and having a reactive functional group at the terminal of at least one side of the second copolymer,
wherein each of the first and fourth repeating units is derived from an ethylenically unsaturated polymerizable monomer having one ethylenic double bond and an alkylene glycol residue, each of the second and fifth repeating units is derived from an ethylenically unsaturated polymerizable monomer having one ethylenic double bond and a functional group which immobilizes a physiologically active substance, and the third repeating unit is derived from an ethylenically unsaturated polymerizable monomer having one ethylenic double bond and a functional group capable of crosslinking.

2: The coating agent composition according to claim 1, wherein the functional group which immobilizes a physiologically active substance is an active ester group.

3: The coating agent composition according to claim 1, wherein the functional group capable of crosslinking is an alkoxysilyl group.

4: The coating agent composition according to claim 1, wherein the reactive functional group is an alkoxysilyl group.

5: The coating agent composition according to claim 1, wherein the ethylenically unsaturated polymerizable monomer having one ethylenic double bond and an alkylene glycol residue is a monomer represented by Formula [1], 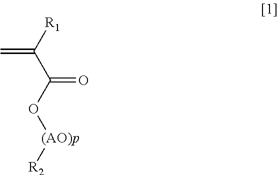
where R₁ represents a hydrogen atom or a methyl group; R₂ represents a hydrogen atom, a methyl group, or an ethyl group; AO represents an alkylene oxide group having 2 to 10 carbon atoms; and p represents the average number of added moles of AO and is a number of 1 to 100.

6: The coating agent composition according to claim 1, wherein the ethylenically unsaturated polymerizable monomer having one ethylenic double bond and a functional group which immobilizes a physiologically active substance is a monomer represented by Formula [2] and having an active ester, 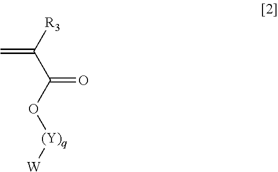
where R₃ represents a hydrogen atom or a methyl group; Y represents AO or an alkylene group having 1 to 10 carbon atoms; AO represents an alkylene oxide group having 2 to 10 carbon atoms; q represents the average number of added moles of AO and is a number of 1 to 100; W represents an active ester group; and in a case in which Y represents an alkylene group, q=1.

7: The coating agent composition according to claim 1, wherein the ethylenically unsaturated polymerizable monomer having one ethylenic double bond and a functional group capable of crosslinking is a monomer represented by Formula [3], 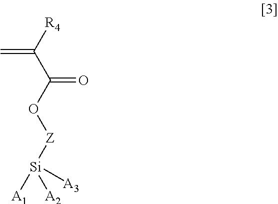
where R₄ represents a hydrogen atom or a methyl group; Z represents an alkylene group having 1 to 20 carbon atoms; and at least one of A₁, A₂ and A₃ represents a group capable of hydrolysis, while the others are inactive groups that are not hydrolyzable.

8: The coating agent composition according to claim 1, wherein the first copolymer is represented by Formula [4], 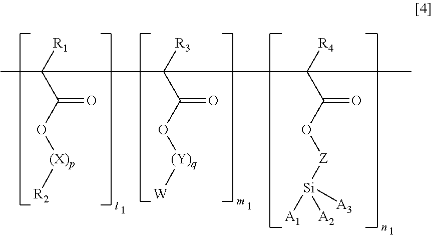
where R₁ represents a hydrogen atom or a methyl group; R₂ represents a hydrogen atom, a methyl group, or an ethyl group; X represents an alkylene glycol residue having 2 to 10 carbon atoms; p represents the number of repetitions of the alkylene glycol residue X and is a number of 1 to 100; R₃ represents a hydrogen atom or a methyl group; Y represents AO or an alkylene group having 1 to 10 carbon atoms; AO represents an alkylene oxide group having 2 to 10 carbon atoms; q represents the average number of added moles of AO and is a number of 1 to 100; W represents an active ester group; in a case in which Y represents an alkylene group, q=1; R₄ represents a hydrogen atom or a methyl group; Z represents an alkylene group having 1 to 20 carbon atoms; at least one of A₁, A₂ and A₃ represents a group capable of hydrolysis, while the others represent inactive groups that are not hydrolyzable; l₁, m₁, and n₁ represent the respective proportions of the repeating units relative to the total number of repeating units; the proportion of l₁ with respect to the sum of l₁, m₁ and n₁ is 5 to 98 mol %, the proportion of m₁ with respect to the sum of l₁, m₁ and n₁ is 1 to 94 mol %; and the proportion of n₁ with respect to the sum of l₁, m₁ and n₁ is 0.01 to 30 mol %.

9: The coating agent composition according to claim 1, wherein the second copolymer is represented by Formula [5], 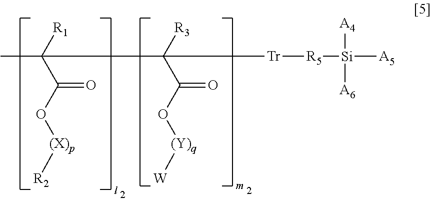
where R₁ represents a hydrogen atom or a methyl group; R₂ represents a hydrogen atom, a methyl group, or an ethyl group; X represents an alkylene glycol residue having 2 to 10 carbon atoms; p represents the number of repetitions of the alkylene glycol residue X and is a number of 1 to 100; R₃ represents a hydrogen atom or a methyl group; Y represents AO or an alkylene group having 1 to 10 carbon atoms; AO represents an alkylene oxide group having 2 to 10 carbon atoms; q represents the average number of added moles of AO and is a number of 1 to 100; W represents an active ester group; in a case in which Y represents an alkylene group, q=1; R₅ represents a hydrocarbon chain having 1 to 20 carbon atoms, which may be interrupted by —O—, —S—, —NH—, —CO—, or —CONH—; at least one of A₄, A₅ and A₆ represents a group capable of hydrolysis, while the others represent inactive groups that are not hydrolyzable; l₂ and m₂ represent the respective proportions of the repeating units relative to the total number of repeating units; the proportion of l₂ with respect to the sum of l₂ and m₂ is 5 to 98 mol %; the proportion of m₂ with respect to the sum of l₂ and m₂ is 1 to 94 mol %; and Tr represents a group derived from a chain transfer agent.

10: A coating agent kit the for coating a surface of a solid-phase substrate, comprising:
a first container containing a first copolymer having a first repeating unit, a second repeating unit, and a third repeating unit; and
a second container containing a second copolymer having a fourth repeating unit and a fifth repeating unit, and having a reactive functional group at the terminal of at least one side of the second copolymer,
wherein each of the first and fourth repeating units is derived from an ethylenically unsaturated polymerizable monomer having one ethylenic double bond and an alkylene glycol residue, each of the second and fifth repeating units is derived from an ethylenically unsaturated polymerizable monomer having one ethylenic double bond and a functional group which immobilizes a physiologically active substance, and the third repeating unit is derived from an ethylenically unsaturated polymerizable monomer having one ethylenic double bond and a functional group capable of crosslinking.

11: A method for producing a solid-phase substrate having a coated surface, comprising:
applying, on a surface of a solid-phase substrate, a coating agent composition comprising a first copolymer, a second copolymer, and a solvent; and
removing the solvent from the coating agent composition applied on the solid-phase substrate,
wherein the first copolymer has a first repeating unit, a second repeating unit, and a third repeating unit, the second copolymer has a fourth repeating unit and a fifth repeating unit, and has a reactive functional group at the terminal of at least one side of the second copolymer, each of the first and fourth repeating units is derived from an ethylenically unsaturated polymerizable monomer having one ethylenic double bond and an alkylene glycol residue, each of the second and fifth repeating units is derived from an ethylenically unsaturated polymerizable monomer having one ethylenic double bond and a functional group which immobilizes a physiologically active substance, and the third repeating unit is derived from an ethylenically unsaturated polymerizable monomer having one ethylenic double bond and a functional group capable of crosslinking.

12: A method for producing a solid-phase substrate having a coated surface, comprising:
applying, on a surface of a solid-phase substrate, a first coating agent composition comprising a first copolymer and a solvent;
removing the solvent from the first coating agent composition applied on the solid-phase substrate such that the solid-phase substrate has the surface coated with the first copolymer;
applying, on the surface of the solid-phase substrate coated with the first copolymer, a second coating agent composition comprising a second copolymer and a solvent, and
removing the solvent from the second coating agent composition applied on the solid-phase substrate such that the solid-phase substrate has the surface coated with the first copolymer and the second copolymer,
wherein the first copolymer has a first repeating unit, a second repeating unit, and a third repeating unit, the second copolymer has a fourth repeating unit and a fifth repeating unit, and has a reactive functional group at the terminal of at least one side of the second copolymer, each of the first and fourth repeating units is derived from an ethylenically unsaturated polymerizable monomer having one ethylenic double bond and an alkylene glycol residue, each of the second and fifth repeating units is derived from an ethylenically unsaturated polymerizable monomer having one ethylenic double bond and a functional group which immobilizes a physiologically active substance, and the third repeating unit is derived from an ethylenically unsaturated polymerizable monomer having one ethylenic double bond and a functional group capable of crosslinking.

13: A substrate, comprising:
a solid-phase substrate;
a first copolymer coated on a surface of the solid-phase substrate and having a first repeating unit, a second repeating unit and, a third repeating unit; and
a second copolymer coated on the surface of the solid-phase substrate and having a fourth repeating unit, a fifth repeating unit, and a reactive functional group at the terminal of at least one side of the second copolymer,
wherein each of the first and fourth repeating units is derived from an ethylenically unsaturated polymerizable monomer having one ethylenic double bond and an alkylene glycol residue, each of the second and fifth repeating units is derived from an ethylenically unsaturated polymerizable monomer having one ethylenic double bond and a functional group which immobilizes a physiologically active substance, and the third repeating unit is derived from an ethylenically unsaturated polymerizable monomer having one ethylenic double bond and a functional group capable of crosslinking.

14: A biosensor, comprising:
the substrate of claim 13; and
a physiologically active substance immobilized on the substrate.

15: A method for producing a biosensor, comprising:
immobilizing a physiologically active substance on the substrate of claim 13.

16: The coating agent composition according to claim 2, wherein the functional group capable of crosslinking is an alkoxysilyl group.

17: The coating agent composition according to claim 2, wherein the reactive functional group is an alkoxysilyl group.

18: The coating agent composition according to claim 2, wherein the ethylenically unsaturated polymerizable monomer having one ethylenic double bond and an alkylene glycol residue is a monomer represented by Formula [1] 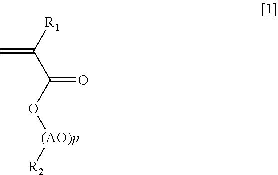
where R₁ represents a hydrogen atom or a methyl group; R₂ represents a hydrogen atom, a methyl group, or an ethyl group; AO represents an alkylene oxide group having 2 to 10 carbon atoms; and p represents the average number of added moles of AO and is a number of 1 to 100.

19: The coating agent composition according to claim 2, wherein the ethylenically unsaturated polymerizable monomer having one ethylenic double bond and a functional group for immobilizing a physiologically active substance is a monomer represented by Formula [2] and having an active ester, 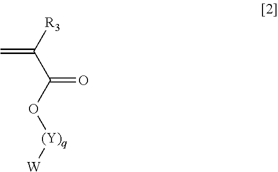
where R₃ represents a hydrogen atom or a methyl group; Y represents AO or an alkylene group having 1 to 10 carbon atoms; AO represents an alkylene oxide group having 2 to 10 carbon atoms; q represents the average number of added moles of AO and is a number of 1 to 100; W represents an active ester group; and in a case in which Y represents an alkylene group, q=1.

20: The coating agent composition according to claim 2, wherein the ethylenically unsaturated polymerizable monomer having one ethylenic double bond and a functional group capable of crosslinking is a monomer represented by Formula [3], 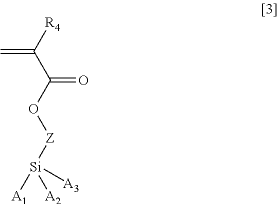
where R₄ represents a hydrogen atom or a methyl group; Z represents an alkylene group having 1 to 20 carbon atoms; and at least one of A₁, A₂ and A₃ represents a group capable of hydrolysis, while the others are inactive groups that are not hydrolyzable.
